(19) United States
(12) Patent Application Publication
(10) Pub. No.: US 2024/0189553 A1
(43) Pub. Date: Jun. 13, 2024

(54) EXPANDABLE BALLOON CATHETER

(71) Applicant: OrbusNeich Medical (Shenzhen) Co., Ltd., Shenzhen, Guangdong (CN)

(72) Inventors: Songyun XU, Shenzhen, Guangdong (CN); Lin WAN, Shenzhen, Guangdong (CN); Jingjing ZHU, Shenzhen, Guangdong (CN); Yirong GONG, Shenzhen, Guangdong (CN); Chang PENG, Shenzhen, Guangdong (CN); Mian LI, Shenzhen, Guangdong (CN); Xueying WEI, Shenzhen, Guangdong (CN)

(73) Assignee: OrbusNeich Medical (Shenzhen) Co., Ltd., Shenzhen, Guangdong (CN)

(21) Appl. No.: 18/554,937

(22) PCT Filed: Apr. 12, 2021
(86) PCT No.: PCT/CN2021/086402

§ 371 Date: Oct. 11, 2023

(51) Int. Cl. A61M 25/10; A61L 29/04; A61L 29/08; A61L 29/16

(52) U.S. Cl. CPC A61M 25/1029; A61L 29/049; A61L 29/085; A61L 29/16; A61M 25/104; A61L 2300/416; A61L 2300/608; A61M 2025/1031; A61M 2025/105; A61M 2025/1075

(57) ABSTRACT

Balloon catheter (22) having a coating comprises a primer layer (24) comprising at least one or more substantially hydrophilic compounds, a first layer (26) comprising an inclusion compound; and a second layer (28) comprising a mixture of a hydrophobic polymer, an active therapeutic agent and the inclusion compound.

### FIELD

The present disclosure relates to an expandable “balloon” catheter used for delivery of a pharmaceutically active agent released upon contact with the walls of a body cavity. In particular, the present disclosure relates to an expandable catheter balloon with an improved coating and a method of coating the same.

### BACKGROUND

The stenosis or reduction/blocking of the luminal diameter of a diseased blood vessel at one or more sites is commonly treated by applying mechanical expansionary forces to the restriction using minimally invasive techniques such as percutaneous transluminal angioplasty (PTA) and other variants.

These techniques typically utilise a small flexible plastic tube or catheter to provide access to a guidewire or another instrument (such as a “balloon” catheter) to access the site of restricted flow. At the appropriate location proximal the restriction in the blood vessel, the distal end of balloon catheter may be inflated for a short period of time to apply an expansionary mechanical force to the walls of the diseased blood vessel before being withdrawn. Optionally a stent may be inserted to maintain the lumen of the vessel after withdrawal of the balloon catheter, although the presence of a stent has been implicated in severe complications which range from acute stent thrombosis to restenosis (reduction in luminal diameter of more than 50%).

The application of expansionary mechanical force to the diseased area initiates a biological response from the tissue, which includes triggering proliferation of dormant smooth muscle cells in the walls of the blood vessel. These smooth muscle cells migrate into the innermost endothelial layers of the blood vessel (tunica media) as part of the healing response. The proliferation and migration of the smooth muscle cells is known as neointimal hyperplasia and together with elastic recoil results in decreased lumen space or restenosis of the vessel. Accordingly, restenosis presents a significant challenge to the effectiveness of angioplasty procedures.

Attempts have been made to address the problems of restenosis. One approach is by using coated balloons which deliver anti proliferative/anti-inflammatory therapeutic agents or drugs at the same time as applying the expansionary force to the site of restricted flow in the diseased vessel.

However, it is necessary to balance many requirements such as maximum therapeutic dosage without toxicity, release profile of the drug, effective transfer of drug during the short contact with vessel wall, and retention of the drug as the balloon catheter is manoeuvred to the site of the procedure. Unfortunately, many of the prior art arrangements are not able to balance these multiple (often conflicting) requirements which in turn means that such devices are ineffective in reducing restenosis.

Accordingly, it is an object of the present disclosure to address or at least ameliorate some of the above problems or at least provide an alternative choice.

### SUMMARY OF THE DISCLOSURE

Features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims.

In accordance with a first aspect of the present disclosure, there is provided a balloon catheter comprising: an expandable portion on an elongated body having a coating extending substantially about the external outer surface of at least the expandable portion; wherein the coating comprising:
a primer layer (24) on the surface of the expandable portion comprising at least one or more substantially hydrophilic compounds,
a first layer (26) substantially overlying the primer layer and comprising an inclusion compound;
a second layer (28) substantially overlying the first layer and comprising a mixture of a hydrophobic polymer, an active therapeutic agent and the inclusion compound.

In one embodiment, the active therapeutic agent of the top layer is selected from the group comprising sirolimus, everolimus, paclitaxel and taxol and including derivatives, hydrates, esters, salts, polymorphs or analogs thereof. In one embodiment, wherein the mass ratio of inclusion compound and hydrophobic polymer is 1:1-1:10,

Preferably, the total loading of the active therapeutic agent of the coating of the surface of the balloon is 1-3 μg/mm²

In one embodiment, the at least one or more hydrophilic compound(s) of the primer layer is/are selected from the group comprising polyurethane acrylate, polyvinylpyrrolidone, polyvinyl alcohol, polyethylene glycol and polyacrylic acid.

In one embodiment, the second layer comprises a homogeneous mixture of the hydrophobic polymer, an active therapeutic agent and the inclusion compound. In an alternative embodiment, the second layer comprises microspheres having the active therapeutic agent encapsulated therein.

Advantangeously, the inclusion compound is chitosan, β-cyclodextrin, hydroxypropyl-β-cyclodextrin, hydroxypropyl-γ-cyclodextrin, hydroxyethyl cellulose, carboxymethyl cellulose, iopromide and derivatives thereof and dextran. The hydrophobic polymer carrier may be selected from polylactic acid, polylactide hexalactide and polyglycolide lactide and has a molecular weight of 1-100 kDa.

In another aspect of the present disclosure, it is provided a method of coating a balloon catheter, which comprising the following steps:
applying to the surface of a balloon catheter a first solution comprising at least one more of the group consisting of from ethanol, acetone, isopropanol, propanol, ethyl acetate, dichloromethane, tetrahydrofuran, acetonitrile and one or more hydrophilic compounds selected from the group consisting of polyurethane acrylate, polyvinylpyrrolidone, polyvinyl alcohol, polyethylene glycol and polyacrylic acid,
curing the coated catheter under hot air or ultraviolet light,
applying to the coated catheter a second solution comprising at least one or more solvents in the group comprising ethanol, water, acetone and isopropanol into which an inclusion compound has been dissolved;
drying the coated catheter and
applying to the coated catheter a third solution comprising a therapeutic compound, and a polymer solution in a solvent, said solvent selected from the group comprising ethanol, water, acetone, isopropanol, dichloromethane, trichloromethane and tetrahydrofuran.

Preferably, the step of applying of the solutions to the catheter is performed by dipping, spraying or brushing the solutions thereon. In one embodiment, microspheres are formed by aggregation of the therapeutic compound, the polymer and the inclusion compound.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore limiting of its scope, the principles herein are described and explained with additional specificity and detail through the use of the accompanying drawings.

Preferred embodiments of the present disclosure will be explained in further detail below by way of examples and with reference to the accompanying drawings, in which:—

FIG. 1A depicts an exemplary catheter balloon having two layers.

FIG. 1B depicts an exemplary balloon in an embodiment of the present disclosure having three layers.

FIG. 1C depicts the exemplary balloons of FIG. 1A or 1B when positioned on a guidewire in an un-expanded state.

FIG. 2A depict exemplary balloons according to the embodiment depicted in FIG. 1A with cyclodextrin directly coated thereon.

FIG. 2B (i)-(ii) depict the exemplary balloons of FIG. 2A after a second layer (drug coating) has been applied on top of the cyclodextrin layer, before folding. The balloons depicted are coated with beta cyclodextrin at concentration of 50 mg/ml (left), and 100 mg/ml (right) and then the balloon was further coated with drug/polymer/dextrin solution.

FIG. 2C(i)-(ii) depict exemplary balloons of the embodiments depicted in FIG. 2A after the cyclo-dextrin and drug coated balloon were folded.

FIG. 3A depicts the balloon prepared according to the schematic arrangement depicted in FIG. 1B with the hydrophilic coating treatment of the first layer and cyclo-dextrin coating of second layer on the inflated balloon outer surface. The surface of the cyclo-dextrin coated balloon is uniform and transparent.

FIG. 38 depicts the balloon of FIG. 3A having the third layer coating of drug/polymer/cyclodextrin applied before folding of the coated balloon,

FIG. 3C depicts the drug/polymer/cyclodextrin balloon of FIG. 3B was folded in an unopened state.

FIG. 3D depicts that the drug/polymer/cyclodextrin coated and folded balloon (shown as FIG. 3C) was inflated as 8 atm pressure.

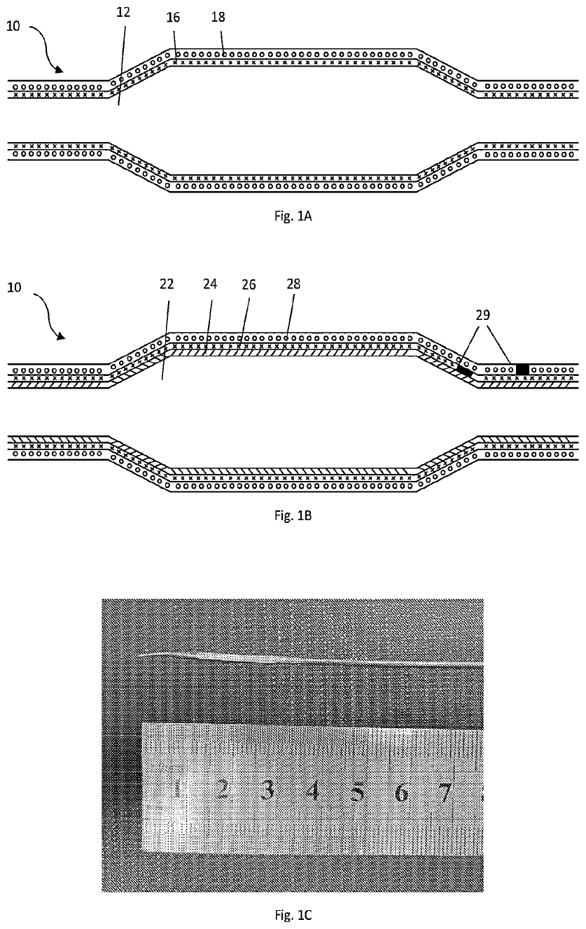

FIG. 4A(i) depicts a drug/polymer/cyclodextrin coated balloon prepared according to the schematic arrangement depicted in FIG. 1B with 66.67% of a weight ratio of sirolimus to polymer PLGA (MW 49700) in the inflated state with 2 atm pressure before folding of the coated balloon.

FIG. 4B(i) depicts the drug/polymer/cyclodextrin coated balloon prepared according to the schematic arrangement depicted in FIG. 1B with 50.0% of a weight ratio of sirolimus to polymer PLGA (MW 49700) in the inflated state with 2 atm pressure before folding of the coated balloon.

FIG. 4C(i) depicts the drug/polymer/cyclodextrin coated balloon prepared according to the schematic arrangement depicted in FIG. 1B with 37.5% of a weight ratio of sirolimus to polymer PLGA (MW 49700) in the inflated state with 2 atm pressure before folding of the coated balloon.

FIG. 4A(ii) depicts the drug/polymer/cyclodextrin coated balloon prepared according to the schematic arrangement depicted in FIG. 1B with 66.67% of a weight ratio of sirolimus to polymer PLGA (MW 49700) prior to coated balloon folding after the coated balloon was folded.

FIG. 4B(ii) depicts the drug/polymer/cyclodextrin coated balloon prepared according to the schematic arrangement depicted in FIG. 1B with 50.0% of a weight ratio of sirolimus to polymer PLGA (MW 49700) after the coated balloon was folded and then the balloon was inflated to 8 atm pressure.

FIG. 4C(ii) depicts the drug/polymer/cyclodextrin coated balloon prepared according to the schematic arrangement depicted in FIG. 1B with 37.5% of a weight ratio of sirolimus to polymer PLGA (MW 49700) after the coated balloon was folded and then the balloon was inflated to 8 atm pressure.

FIG. 5A(i) depicts the drug/polymer/cyclodextrin coated balloon prepared according to the schematic arrangement depicted in FIG. 1B with 50.0% of a weight ratio of sirolimus to polymer PLGA (MW 14300) in the inflated state with 2 atm pressure before folding of the coated balloon.

FIG. 5B(i) depicts the drug/polymer/cyclodextrin coated balloon prepared according to the schematic arrangement depicted in FIG. 1B with 44.44% of a weight ratio of sirolimus to polymer PLGA (MW 14300) in the inflated state with 2 atm pressure before folding of the coated balloon.

FIG. 5C(i) depicts the drug/polymer/cyclodextrin coated balloon prepared according to the schematic arrangement depicted in FIG. 1B with 37.5% of a weight ratio of sirolimus to polymer PLGA (MW 14300) in the inflated state with 2 atm pressure before folding of the coated balloon.

FIG. 5A(ii) depicts the drug/polymer/cyclodextrin coated balloon prepared according to the schematic arrangement depicted in FIG. 1B with 50.0% of a weight ratio of sirolimus to polymer PLGA (MW 14300) after the coated balloon was folded and then the balloon was inflated to 8 atm pressure.

FIG. 5B(ii) depicts the drug/polymer/cyclodextrin coated balloon prepared according to the schematic arrangement depicted in FIG. 1B with 44.4% of a weight ratio of sirolimus to polymer PLGA (MW 14300) after the coated balloon was folded and then the balloon was inflated to 8 atm pressure.

FIG. 5C(ii) depicts the drug/polymer/cyclodextrin coated balloon prepared according to the schematic arrangement depicted in FIG. 1B with 37.5% of a weight ratio of sirolimus to polymer PLGA (MW 14300) after the coated balloon was folded and then the balloon was inflated to 8 atm pressure.

FIG. 6A depicts a drug/polymer/cyclodextrin coated balloon prepared according to the schematic arrangement depicted in FIG. 1B with 66.67% of a weight ratio of paclitaxel to polymer PLGA (MW 49700) in the inflated state with 2 atm pressure before folding of the coated balloon.

FIG. 6B depicts the drug/polymer/cyclodextrin coated balloon prepared according to the schematic arrangement depicted in FIG. 1B with 66.67% of a weight ratio of paclitaxel to polymer PLGA (MW 49700) after the coated balloon was folded.

FIG. 6C depicts the drug/polymer/cyclodextrin coated balloon prepared according to the schematic arrangement depicted in FIG. 1B with 66.67% of a weight ratio of paclitaxel to polymer PLGA (MW 49700) after the coated balloon was folded and then the balloon was inflated to 8 atm pressure.

FIG. 7A (i) displays the drug 6.52% loss of drug/polymer/cyclodextrin coated balloon with hydrophilic coating treatment of balloon surface after folding process of a balloon embodiment prepared according to the schematic arrangement depicted in FIG. 1B.

FIG. 7A (ii) displays the drug 43.96% loss of drug/polymer/cyclodextrin coated balloon without hydrophilic coating treatment of balloon surface after folding process of a balloon embodiment prepared according to the schematic arrangement depicted in FIG. 1B.

FIG. 7B displays the drug loss ratio of drug/polymer/cyclodextrin coated balloon with the 66.67%, 50.0%, 37.5% of a weight ratio of sirolimus to polymer PLGA (MW 49700) before/after folding coated balloon prepared according to the schematic arrangement depicted in FIG. 1B.

FIG. 7B also displays the drug loss ratio of drug/polymer/cyclodextrin coated balloon with the 50.0%, 44.4%, 37.5% of a weight ratio of sirolimus to polymer PLGA (MW 14300) before/after folding coated balloon prepared according to the schematic arrangement depicted in FIG. 1B.

FIG. 8A depicts the drug elution rate of 66.67%, 50.0%, 37.5% of a weight ratio of sirolimus to polymer PLGA (MW 49700) coated balloon prepared according to the schematic arrangement depicted in FIG. 1B against time in the PBS buffer solution at 37 centigrade with 120 RPM shaking. As result shown, 66.67% of a weight ratio of sirolimus to polymer PLGA (MW 49700) coated balloon showed the best elution rate.

FIG. 8B depicts the drug elution rate of 50.0%, 44.4%, 37.5% of a weight ratio of sirolimus to polymer PLGA (MW 14300) coated balloon prepared according to the schematic arrangement depicted in FIG. 1B against time in the PBS buffer solution at 37 centigrade with 120 RPM shaking.

FIG. 9A depicts a particle size distribution of 66.67% of a weight ratio of sirolimus to polymer PLGA (MW 49700), PLGA (MW 14300) and PLA (MW15500) coated balloon surface prepared according to the schematic arrangement depicted in FIG. 1B by SEM analysis.

FIG. 9B depicts the particle size distribution of the 66.67% of a weight ratio of sirolimus to polymer PLGA (MW 49700) coated balloon prepared according to the schematic arrangement depicted in FIG. 1B analyzed by Scanning Electron Micrograph.

FIG. 10A depicts the visual example of the 66.67% of a weight ratio of sirolimus to polymer PLGA (MW 49700) coated balloon prepared according to the schematic arrangement depicted in FIG. 1B with folding process at 20 times magnitude.

FIG. 10B depicts the visual example of the 66.67% of a weight ratio of sirolimus to polymer PLGA (MW 49700) coated balloon prepared according to the schematic arrangement depicted in FIG. 1B with inflating at 8 atm pressure.

FIG. 10C(i) depicts the 66.67% of a weight ratio of sirolimus to polymer PLGA (MW 49700) coated balloon as illustrated in FIG. 9B at 50 times magnification.

FIG. 10C(ii) depicts the 66.67% of a weight ratio of sirolimus to polymer PLGA (MW 49700) coated balloon as illustrated in FIG. 9B at 200 times magnification.

FIG. 11A depicts an exemplary experimental arrangement of simulating in vivo conditions with guiding catheter and silica vessel to evaluate the transferred drug content from the 66.67% of a weight ratio of sirolimus to polymer PLGA (MW 49700) coated balloon to silica vessel.

FIG. 11B is an enlarged view of the artificial silica vessel used in the experimental setup depicted in FIG. 10A.

FIG. 11C(i) depicts a simulated blood vessel (silica vessel) with drug/polymer transferred from an exemplary balloon prepared according to the schematic arrangement depicted in FIG. 1B at 20 times magnification.

FIG. 11C(ii) depicts a simulated blood vessel (silica tube) with drug/polymer transferred from an exemplary balloon prepared according to the schematic arrangement depicted in FIG. 1B at ×100 magnification.

FIG. 12A is a series of measurements simulating the conditions of the drug content in a simulated blood vessel determined using the experimental arrangement of FIG. 11A and a balloon produced according to the arranged depicted in schematic representation FIG. 1B.

FIG. 12B is a further a series of measurements simulating the conditions of the drug content in a simulated blood vessel determined using the experimental arrangement of FIG. 11A and balloons produced according to the arrangement depicted in schematic representation FIG. 1B with varying combinations of compounds.

### DETAILED DESCRIPTION OF THE PREFERRED EMBODIMENTS

Various embodiments of the disclosure are discussed in detail below. While specific implementations are discussed, it should be understood that this is done for illustration purposes only. A person skilled in the relevant art will recognize that other components and configurations may be used without departing from the scope of the present disclosure.

The disclosed technology addresses the need in the art for an improve coating for a balloon catheter which balances various requirements

As used herein, the term “a pharmaceutically active agent” is interchangeable with the terms “a pharmaceutical agent” or “drug”.

Diseases that can be treated with the present balloon, include, but are not limited to, both coronary artery and peripheral artery diseases as well as others. Non-limiting examples of the diseases or conditions that can be treated by the present balloon or method include atherosclerosis, coronary artery atherosclerosis disease (CAD), peripheral artery atherosclerosis disease (PAD), narrowing of an artery, etc.

Atherosclerosis is one of the leading causes of death and disability in the world. Atherosclerosis involves the deposition of fatty plaques on the luminal surface of arteries. The deposition of fatty plaques on the luminal surface of the artery causes narrowing of the cross-sectional area of the artery. Ultimately, this deposition blocks blood flow distal to the lesion causing ischemic damage to the tissues supplied by the artery. Coronary arteries supply the heart with blood. Coronary artery atherosclerosis disease (CAD) is among the most common, serious, chronic, life-threatening illness in the United States. According to the Centers for Disease Control, 370,000 people die annually from CAD and 735,000 Americans have a heart attack or myocardial infarction (https://www.cdc.gov/heartdisease/facts.htm, retrieved, Feb. 5, 2017). Narrowing of the coronary artery lumen causes destruction of heart muscle resulting first in angina, followed by myocardial infarction and finally death.

Narrowing of the arteries can occur in vessels other than the coronary arteries, including carotid, aortoiliac, infrainguinal, distal profunda femoris, distal popliteal, tibial, subclavian and mesenteric arteries. The prevalence of peripheral artery atherosclerosis disease (PAD) depends on the particular anatomic site affected as well as the criteria used for diagnosis of the occlusion, but as many 8.5 million people in the United States are estimated to suffer from PAD (https://www.cdc.gov/dhdsp/data_statistics/fact sheets/fs_pad.htm, retrieved, Feb. 5, 2017). Traditionally, physicians have used the test of intermittent claudication to determine whether PAD is present.

The present invention also provides for a method of treating any body cavity by releasing a pharmaceutically active agent from an inflatable balloon through an expandable cover into body cavities other than vascular spaces. For example, the genitourinary system, including, the urethra, bladder, ureters, penis and vagina, gastrointestinal system, such as the esophagus, stomach, small intestine or colon, the respiratory system, including, the trachea, bronchi and alveoli can be treated with the balloon of the present invention. Vascular spaces other than coronary arteries may also be treated, including, the aorta, vena cava (inferior and superior) or neurovascular arteries, e.g., carotid arteries, basilar arteries. The coated balloon of the present invention may also be used to create a cavity within a potential space in the body, e.g., muscle, vascular intima or fibrotic tissue. The pharmaceutically active agent is then released into the new body cavity created from the potential space, e.g., within a muscle.

Other diseases may be treated with the coated balloon of the present invention, include inflammatory diseases and cancers. Cancers that can be treated with coated balloon of the present invention include, but are not limited to, bladder, lung cancer, ear, nose and throat cancer, leukemia, colon cancer, melanoma, pancreatic cancer, mammary cancer, prostate cancer, breast cancer, hematopoietic cancer, ovarian cancer, basal cell carcinoma, biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia including acute myeloid leukemia, acute lymphoid leukemia, chronic myeloid leukemia, chronic lymphoid leukemia; liver cancer; lymphoma including Hodgkin's and Non-Hodgkin's lymphoma; myeloma; fibroma, neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

Inflammatory diseases, include, but are not limited to, rheumatoid arthritis, systemic lupus eythematosis (SLE), Crohn's diseases or other collagen vascular diseases. Infectious diseases resulting from bacteria, viruses or prions may also be treated with the balloon of the present invention.

As used herein, w/w means the weight of the pharmaceutically active agent released at any time, t, over total weight of pharmaceutically active agent coated on the balloon; % w/w means w/w×100.

The present invention may be used with any balloon catheter stent delivery system, including balloon catheter stent delivery systems described in U.S. Pat. Nos. 6,168,617, 6,222,097; 6,331,186; 6,478,814; 7,169,162 or 20090254064. Balloon catheters such as those described in U.S. Patent Pub. No. 20040006359 may also be used with the methods of the present invention.

The coating can be applied to a balloon either after the balloon has been compacted for insertion or before insertion. The balloon is compacted by, e.g., crimping or folding. U.S. Pat. Nos. 5,350,361, 7,308,748 or 7,152,452. The balloon is delivered to the intervention site by a delivery device such as a catheter.

Balloons can be delivered, removed, and visualized during delivery and/or removal by methods well known in the art, see, e.g., U.S. Pat. No. 6,610,013 or 7,171,255. The balloons of the present invention can include, compliant (expand, e.g., 16-40%, when pressurized), semi-compliant (expand, e.g., 7-16%, when pressurized), and non-compliant balloons (expand, e.g., 2-7%, when pressurized). The various characteristics, e.g., maximum distensions, i.e. distension from nominal diameter to burst, vary and are well known in the art. Cutting balloons which are also used in angioplasty may be used with the methods and devices of the present invention. The balloon is inflated to a set inflation pressure which is determined by the operator depending on the site and type of balloon. The “rated burst pressure” or “RBP” of the balloon is the maximum guaranteed pressure to which a balloon can be inflated without failing.

Optionally, the balloon may be coated with a lubricant coating before or after application of the pharmaceutically active agent to reduce the coefficient of friction between the pharmaceutically active and the balloon, i.e., sticking. The lubricant coating may be a hydrophilic or hydrophobic coat. Examples of lubricants to reduce the coefficient of friction used in medical devices include: silicone; colloidal solution of water and lecithin; polyphenyl ethers as electrical connector lubricants; and the solid lubricants molybdenum disulphide, PTFE or powdered graphite and boron nitride. The friction coefficients may be reduced as low as 0.001 or less. Alternatively, polymers having non-sticky surfaces can be produced by using a surface modifying compound such as Teflon®, fluoro-containing polymers and copolymers, and the like with vinyl terminal or side groups for chemical solvent resistance and non-sticky surfaces. Polymers having hydrophilic surfaces can be produced by using a surface modifying compound such as polyvinylpyrrolidone, PVA, PEG, and the like. In addition, polymers having a low surface friction can be produced by using a surface modifying compound such as polyvinylpyrrolidone, PVA, PEG, Teflon®, and the like.

In the schematic embodiments depicted in FIG. 1A and FIG. 1B it can be seen that there is depicted a medical device 10 comprising an expandable balloon catheter 12.

Referring to the embodiment depicted schematically in FIGS. 1A and 1B, first layer 16, 26 is applied to the external surface 12, 22 of the balloon. The first layer 16, 26 contains an inclusion compound which is selected from the group consisting of chitosan, β-cyclodextrin, hydroxypropyl-β-cyclodextrin, hydroxypropyl-γ-cyclodextrin, hydroxyethyl cellulose, carboxymethyl cellulose and dextran. However in the examples discussed further below the first layer comprises R-cyclodextrin, hydroxypropyl-β-cyclodextrin, or hydroxypropyl-γ-cyclodextrin.

Applied on top of this layer 16, 26 by dipping, spraying or brushing solutions thereon is a mixture of a hydrophobic polymer, an active therapeutic agent and the inclusion compound of the second layer 18, 28. Preferably the hydrophobic polymer carrier is selected from polylactic acid, polylactide hexalactide and polyglycolide lactide and has a molecular weight of 1-100 kDa, more preferably 50 kDa.

In an alternative embodiment, the top layer comprising iopromide as an inclusion compound is applied as a mixture of hydrophobic polymer, an active therapeutic agent and iopomide. In this embodiment, iopromide is present as the inclusion compound of the second layer. It is expected that the same function as cyclodextrin can be achieved.

Advantageously, the active therapeutic agent of this layer is selected from the group comprising sirolimus, everolimus, paclitaxel and taxol and including pharmaceutically acceptable derivatives, hydrates, esters, salts, polymorphs or analogs thereof.

Preferably the mass ratio of inclusion compound and hydrophobic polymer is 1:1-10:1, more preferably 1-3:1, for example, the ratio can be 3-1.

Advantageously, the total loading of the active therapeutic agent of the surface of the balloon is 0.1-20 μg/mm², preferably 1-3 μg/mm², more preferably 1-2 μg/mm², and most preferably 1.7 μg/mm².

As further discussed below, the drug loading of 1.7 μg/mm² is able to achieve the best result, whereby the drug-coated balloon was found to result in minimum drug loss during the delivery process, and the drug can be effectively transferred to the simulated blood vessel wall (over 40%), leaving behind minimal residual drug on the balloon.

In the embodiment depicted in FIG. 1B, an additional layer, a hydrophilic primer layer 24 is applied to at least a substantial portion of the external surface of the expandable portion 12. Advantageously, the primer layer 24 is a coating which comprises a hydrophilic material selected from polyurethane acrylate, polyvinylpyrrolidone, polyvinyl alcohol, polyethylene glycol and polyacrylic acid.

Applied to substantially overlie the primer coating 24 in the embodiment depicted in FIG. 1B, the first layer 26 is applied by methods known to persons skilled in the art including dipping, spraying or brushing solutions thereon. Advantageously, the first layer contains an inclusion compound which is selected from the group consisting of chitosan, β-cyclodextrin, hydroxypropyl-β-cyclodextrin, hydroxypropyl-γ-cyclodextrin, hydroxyethyl cellulose, carboxymethyl cellulose and dextran. However in the examples discussed further below the first layer is β-cyclodextrin, hydroxypropyl-β-cyclodextrin, hydroxypropyl-γ-cyclodextrin.

It is believed that, with the presence of the hydrophilic primer, cyclodextrin assists the formation of particles of drugs/polymers of the top layer on the surface of the balloon. The size of these polymer/drug particles are large enough that they adhere to the surface of the balloon and adhere with the balloon without being washed away by blood as it is maneuvered to the treatment site. Due to compression by blood vessels at the treatment site and upon expansion of balloon, the drug can be effectively transferred to the blood vessel walls.

A top or second coating layer 28 may be applied by dipping, spraying or brushing solutions thereon to substantially overlie the second layer. Preferably the second layer comprises a mixture of a hydrophobic polymer, an active therapeutic agent and the inclusion compound of the first layer. Preferably the hydrophobic polymer carrier is selected from polylactic acid, polylactide hexalactide and polyglycolide lactide and has a molecular weight of 1-100 kDa.

Advantageously, the active therapeutic agent of the top layer is selected from the group comprising sirolimus, everolimus, paclitaxel and taxol and including derivatives, hydrates, esters, salts, polymorphs or analogs thereof.

Preferably the mass ratio of inclusion compound and hydrophobic polymer is 1:1-10:1, more preferably 3-1.

Advantageously, the total loading of the active therapeutic agent of the surface of the balloon is 0.1-20 μg/mm², preferably 1-3 μg/mm², more preferably 1-2 μg/mm², and most preferably 1.7 μg/mm².

The active therapeutic agents that can be included in the present invention are preferably small molecules that are hydrophobic in nature. For example, one of the preferred therapeutic agents, sirolimus, is a hydrophobic small molecule that can inhibit the proliferation of smooth muscle in blood vessels and prevent blood vessels from being narrowed. The selected hydrophobic polymer is a large molecule that enables the slow release of the active therapeutic agent in blood vessels.

Both the therapeutic agent and the polymer are hydrophobic compounds, allowing them to co-dissolve and form particular compounds. On the other hand, typically, the inclusion compound (e.g. cyclodextrin) is hydrophilic in nature and does not normally dissolve along with therapeutic agent and the polymer. The presence of the inclusion compound assists the formation of particles by the polymers and active therapeutic agent in vivo. The three compounds can be dissolved together, only in a special solvent system in vivo (e.g. one that comprises of ethanol, dichloromethane and water). If the solvent volatilizes and the balance is interrupted, the polymer and therapeutic agent would be separated from the cyclodextrin.

The selected inclusion compounds, including cyclodextrin, display great solubility in blood or buffer salt solutions at 37° C., and therefore can dissolve quickly when placed inside human body. Accordingly, the mixture of polymer and drug that stay on the surface of the balloon can easily transfer from the balloon to the blood vessel wall upon contact with and especially upon compression from blood vessels, thereby exerting the therapeutic effect of the drug.

It is envisaged that, in the presence of inclusion compound (e.g. hydrophilic cyclodextrin), the inclusion compound, the active therapeutic agent and the hydrophobic polymer together form a homogeneous solution. Upon volatilization of the organic solvent (which may be ethanol and/or dichloromethane) and water, the hydrophobic polymer and the active pharmaceutical molecule (drug) aggregate together to form spheres of various sizes.

It is believed that, the polymer and the drug (which would normally form a “cake-like”, or “bamboo shoot-like” structure) are present as small particles due to the presence of cyclodextrin. The sphere-shaped particles formed following aggregation are shown in FIG. 9B, and they are able to rapidly dissolve in water, blood, or buffer salt solutions. These novel features of the drug/inclusion compound/polymer aggregates allow the transfer of the drug to the blood vessel wall, such that the drug's inhibitory effect on smooth muscle can be achieved.

### EXAMPLES

### Example 1 Appearance of Comparative Balloon without a Primer Layer

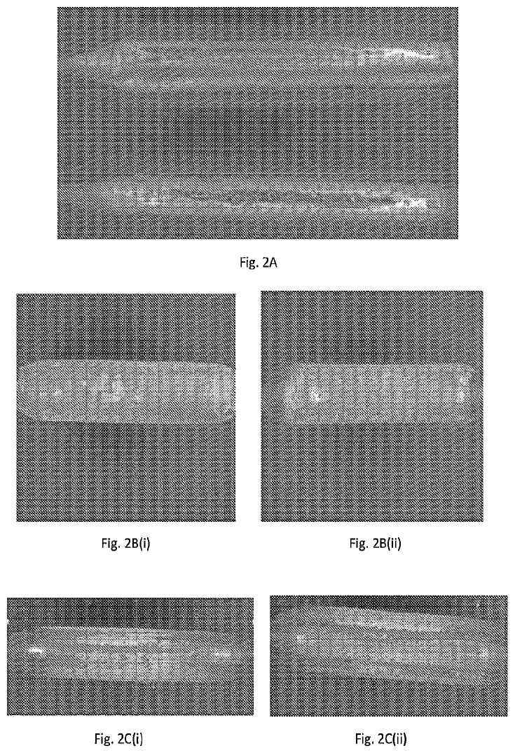

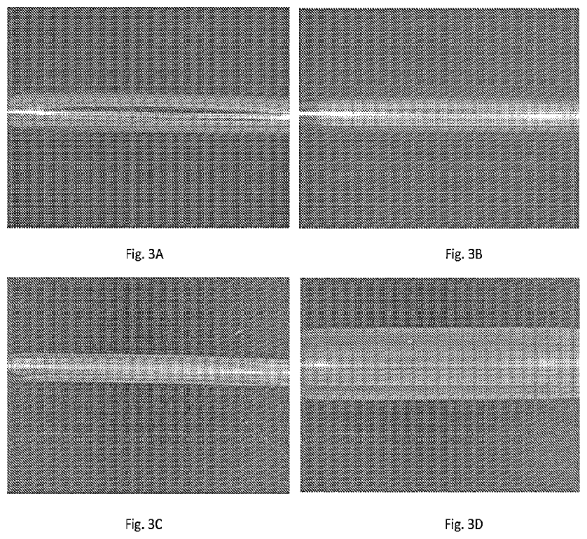

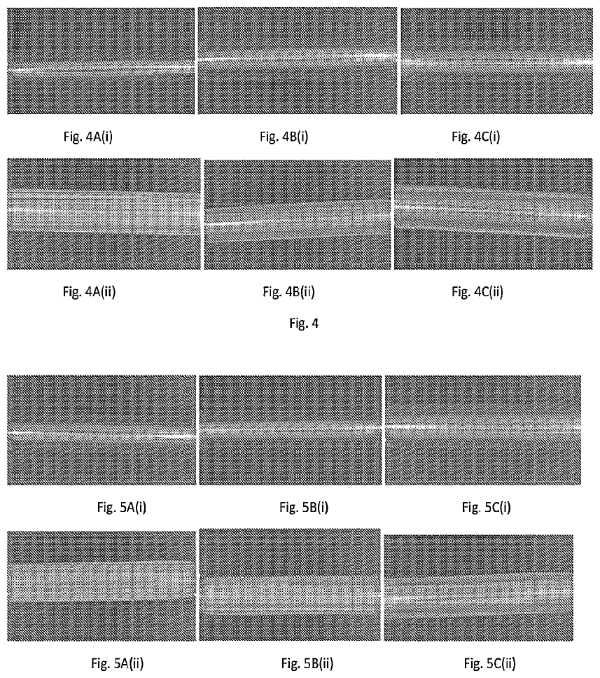

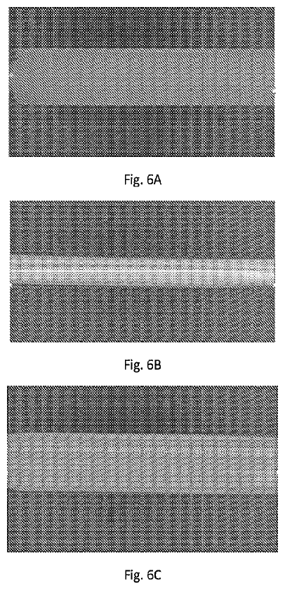

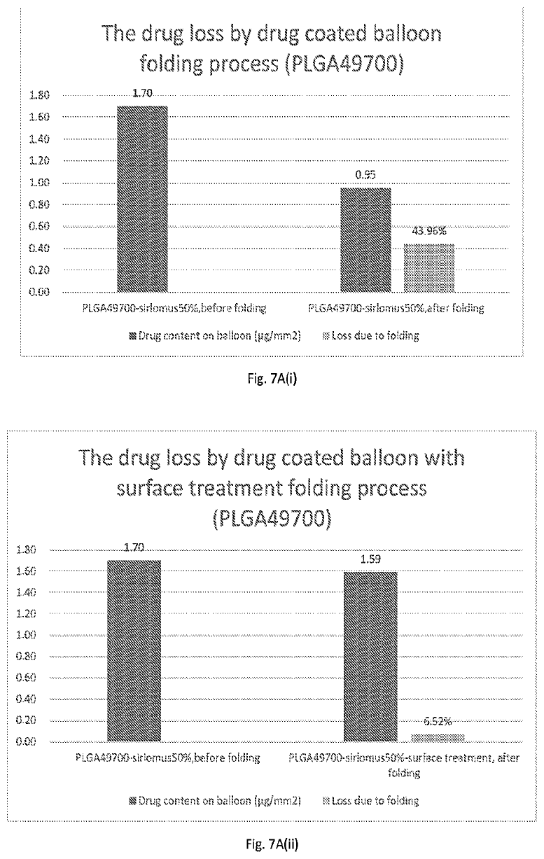

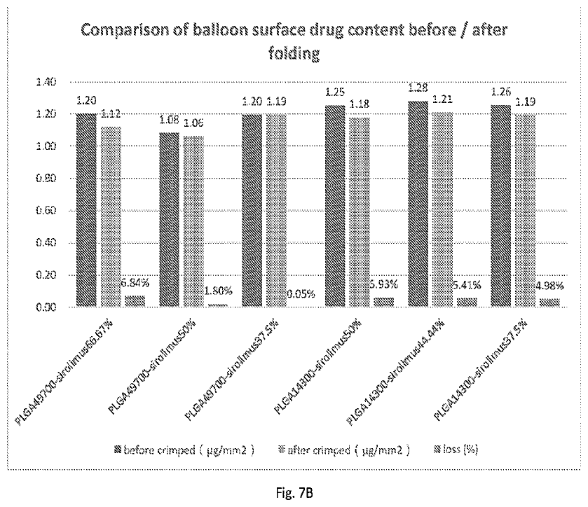

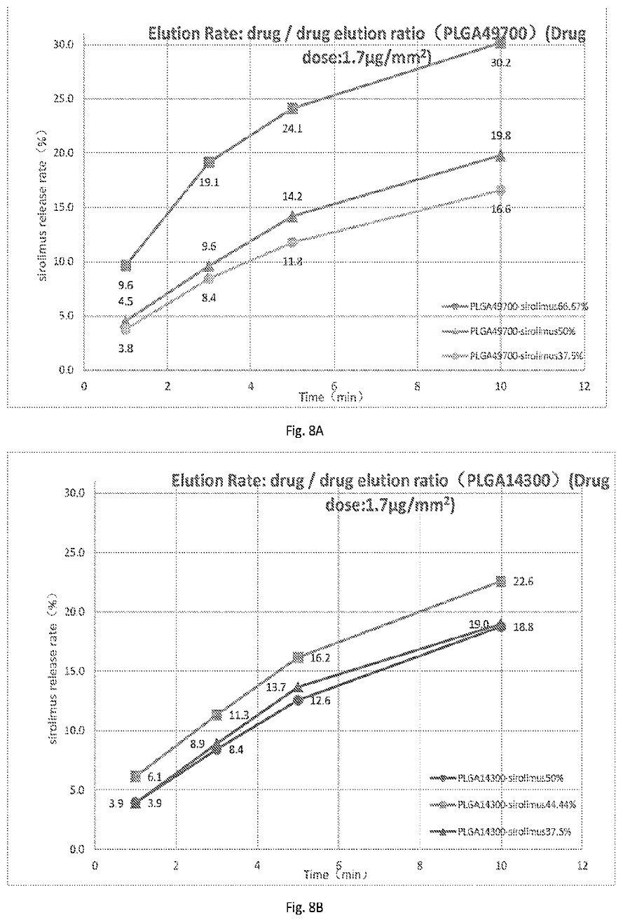

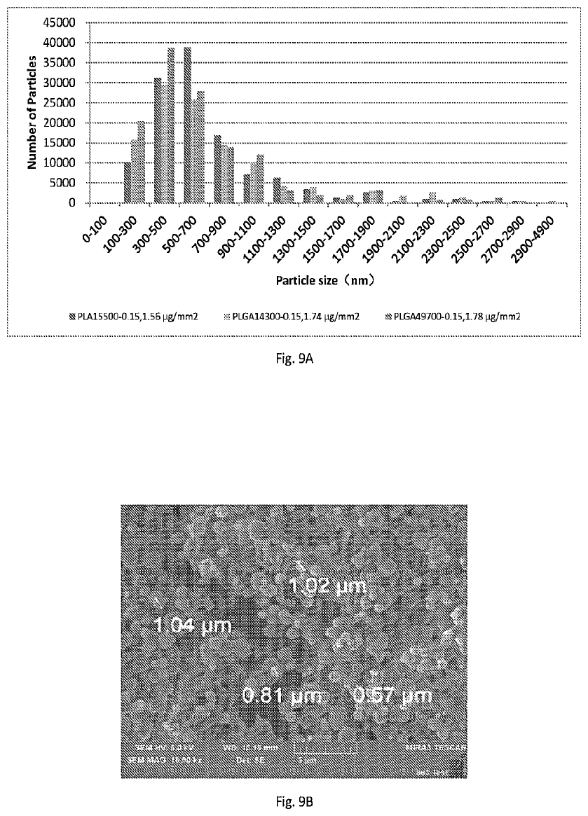

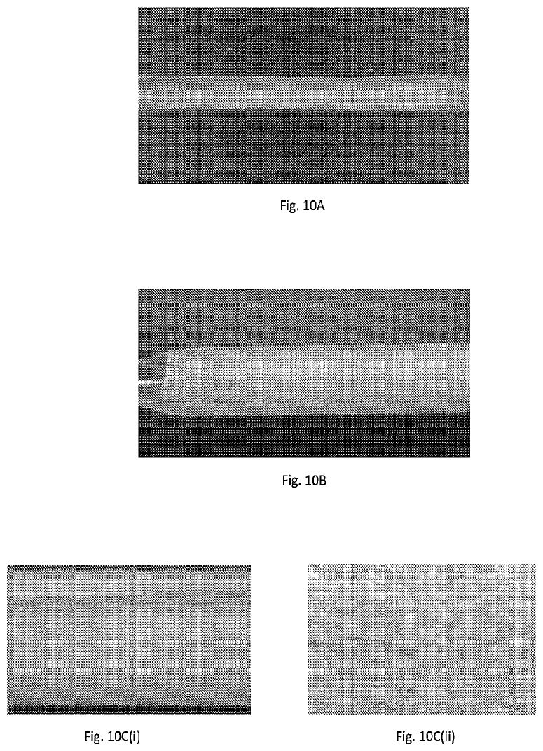

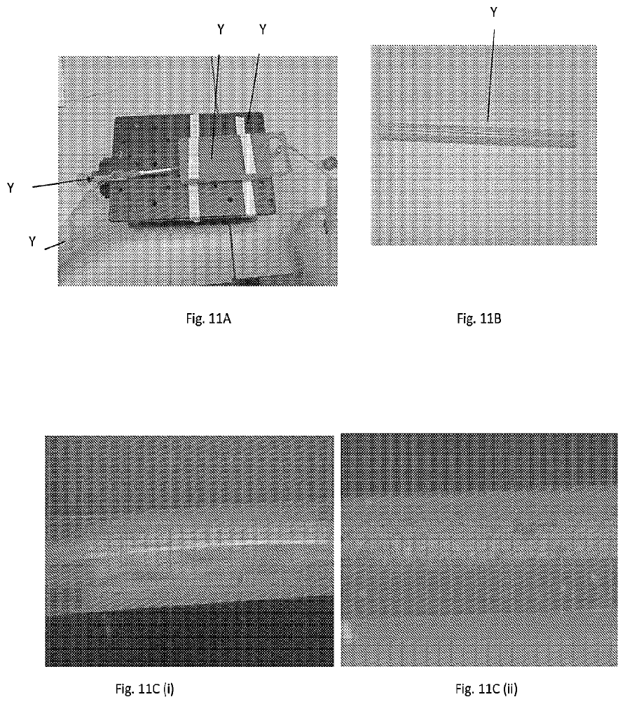

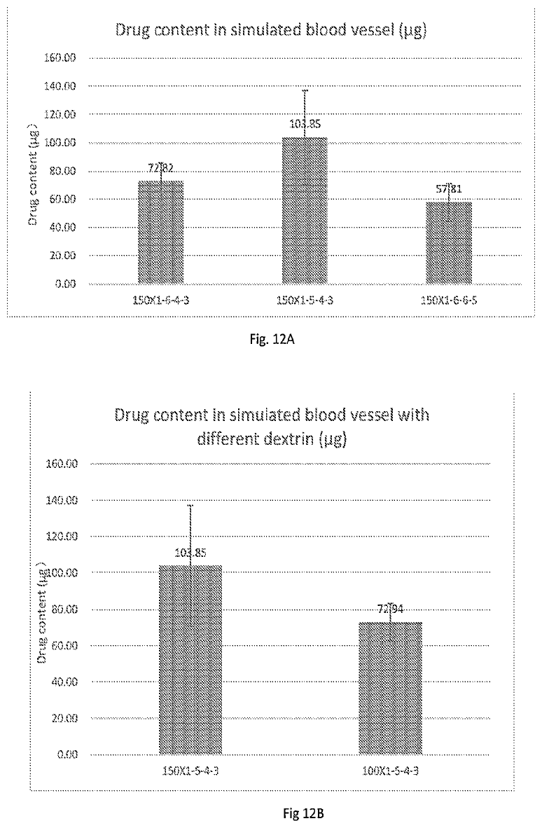

The balloons of FIG. 2A were coated with Beta cyclodextrin directly (i.e. without hydrophilic coating treatment applied to the surface of the balloon) in an embodiment according to FIG. 1A. At this stage these balloons are shown without a drug coating being applied on top of the cyclodextrin layer.

As depicted in FIG. 2A the upper balloon 32 is coated with beta cyclodextrin at a concentration of 50 mg/ml while the lower balloon is coated with beta cyclodextrin at a concentration of 100 mg/ml. It can be seen from FIG. 2A that beta cyclodextrin is not evenly distributed on these balloons, when beta cyclodextrin is directly coated onto the balloon. As depicted in FIG. 2B (i), FIG. 2B (ii) the visual appearance of the catheter balloon after coating with first coating layer of beta cyclodextrin and a top or second layer comprising a drug/polymer and beta cyclodextrin is visible at differing concentrations of the initial coating 50 mg/ml, 100 mg/ml respectively.

Exemplary balloons of the embodiments depicted in FIG. 1A prepared with PLGA49700-sirolimus and PLGA14300-sirolimus after folding are shown in FIG. 2C. The balloons are prepared with first 50 mg/ml beta-cyclodextrin coating and second sirolimus/polymer PLGA/cyclo-dextrin solution (FIG. 2C(i)) and first 100 mg/ml beta-cyclodextrin coating and second sirolimus/polymer PLGA/cyclo-dextrin solution (FIG. 2C(ii)), after the cyclo-dextrin and drug coated balloon were folded.

It can be seen that coating of drug/polymer/cyclodextrin on the balloon was cracked by the folding and un-folding process and a significant amount of drug/polymer/cyclo-dextrin coating has been lost.

### Example 2 Appearance of Balloon Having a Primer Layer

Referring to FIG. 3, there is depicted in more detail the various stages of balloon in accordance with the embodiment depicted in FIG. 1B.

As depicted in FIG. 3A, substantial portions of the balloon have been coated with a hydrophilic coating containing polyvinylpyrrolidone treatment. The composition of the coating is described in US20200339906A1 (herein incorporated by reference).

Next, as depicted in FIG. 3B a coating of Sirolimus (at a drug concentration of 1.7 μg/mm²) was applied to the external surface of the balloon to cover a substantial portion of the balloon. The surface of the balloon coated with drug/polymer/cyclodextrin solution is uniform, flat and translucent. The drug concentration on the balloon was determined by high-performance liquid chromatography. Specifically, the total amount of drug of the balloon was measured by HPLC, and then divided by the total surface area of the balloon to obtain the concentration of the drug on the surface of the balloon. The balloon of FIG. 3B was then folded in an unexpanded state in on itself as shown in FIG. 3C by a standard automatic folding device such as MSI FFS1075S. The balloon was then expanded as shown in FIG. 3D. It can be seen that notwithstanding the folding and subsequent expansion of the balloon that a significant amount of the drug coating is retained on the balloon, notwithstanding some cracks visible on the drug/polymer/cyclodextrin coating surface on the balloon. The drug/polymer/cyclodextrin coating remains uniform in shape and adhere strongly onto the surface of the balloon.

The following table provides measurements of the drug retained on various balloons prepared according to the embodiment depicted in FIG. 1B demonstrating that there is not a significant loss of drug even after folding.

| | Drug dosage on balloon (μg) | | | | | Average | | |
|---|---|---|---|---|---|---|---|---|
| Groups | 1 | 2 | 3 | 4 | 5 | (μg) | SD | RSD(%) |
| Unfolded | 203.40 | 204.73 | 201.32 | | | 203.15 | 1.40 | 0.69 |
| Machine roll | 199.52 | 202.09 | 201.84 | 200.68 | 203.13 | 201.45 | 1.24 | 0.62 |
| after folding | | | | | | | | |
| Manual roll | 205.17 | 203.28 | 204.08 | 204.27 | 202.82 | 203.93 | 0.82 | 0.40 |
| after folding | | | | | | | | |

### Example 3 Appearance of Balloons Coated with Sirolimus and Paclitaxel

Using standard optical microscopy techniques, images depicted in FIGS. 3A-3C and 4A-4C and FIG. 5A-5C for Sirolimis and FIG. 6A-C for Paclitaxel were captured at 20:20 magnification.

As depicted in FIG. 4A(i)-4C(i) pictures of various balloons with concentrations of sirolimus including 66.67%, 50%, 37.5% together with PLGA 49700 when prepared according to embodiment as shown in FIG. 1B have been presented in an expanded state. FIG. 4A(ii)-FIG. 4D(ii) depicts the balloon after it has been folded and re-expanded. It can be seen that substantial portion of the coating has been retained from visual inspection.

Similarly, as depicted in FIG. 5A(i)-5C(i) pictures of various balloons with concentrations of sirolimus including 50%, 44.44%, 37.5% together with PLGA 14300 when prepared according to embodiment as shown in FIG. 1B have been presented in an expanded state. FIG. 5A(ii)-FIG. 5C(ii) depicts the balloon after it has been folded and re-expanded. It can be seen that substantial portion of the coating has been retained from visual inspection.

FIG. 6A-C show various photos of a drug/polymer/cyclodextrin coated balloon prepared according to the schematic arrangement depicted in FIG. 1B with 66.67% of a weight ratio of pacitaxel to polymer PLGA (MW 49700) at different stages: folding (FIG. 6A); un-folding (FIG. 6B) and from folding to deflating (FIG. 6C).

The results confirm that the presence of the hydrophilic primer layer facilitates even distribution of drug/polymer throughout the surface of the balloons shown.

A frequency distribution of the size of particles formed of polymers of various molecular weights (PLA15500, PLGA 4300, PLGA49700) and different concentration of drug as found on the balloon is illustrated in FIG. 9A upon a Scanning Electron Micrograph (SEM) analysis particle counting instrument. It is found that the particles on the balloon are in accordance with the requirements of CFDA regulations. This analysis is performed in accordance on the balloons depicted in FIG. 1B (with the specified concentrations of sirolimus and Polymer).

| Sample Batch No. | | 7400212011 |
|---|---|---|
| | | (3.25 × 15) |
| Number of test samples (pcs) | | 3 |
| Blank control (N_{b}) | | 0 |
| Contamination index of particles in the eluents | | 41 |
| of 3 samples pool (N) | | |
| Acceptance | N_{b} | ≤1.8 |
| criteria | N | ≤270 |

| | |
|---|---|
| Test Result | Pass |

Acceptance criteria: The number of particles in the 100 mL blank control test should meet Nb≤1.8; the contamination index of 3 samples should satisfy N≤5270.

Result: The contamination index of the three samples measured by the WI-00414 method was 41, which was less than the acceptance standard of 270, showing that all three samples have passed.

The SEM analysis were performed after coated balloon was immersed in the PBS solution with shaking speed 120 RPM for 2 mins at 37 centigrade. As shown in FIG. 9A, 66.67% weight ratio of sirolimus to polymer PLGA (MW 49700) coated balloon showed the best particle size distribution at 300-500 nm size.

A Scanning Electron Micrograph of a balloon prepared according to the schematic arrangement depicted in FIG. 1B is provided in FIG. 9B. The particles of the drug/polymer are formed in a suitable size that allows better adherence to the balloon.

### Example 4—Effect of Folding on Balloon State

As is known in the art, once a balloon with a drug eluting coating has been prepared, it is necessary to fold the balloon to minimize the balloon profile and also protect it from damage/cross contamination. Typically this operation is performed by an automatic machine such as MSI FFS1075S drug-eluting balloon pleating and folding equipment or a similar equipment.

Unfortunately the folding of the balloon is yet another stage that the drug coating may be damaged, and accordingly it is important that coating applied to the balloon is substantially retained on the balloon even after folding has been performed. This can be verified by optical inspection or other analytical techniques as is discussed further below.

The effect of folding on balloon prepared with and without surface treatment is illustrated in this example. Balloons are prepared with coatings prepared by a mixture of PLGA49700 polymer and sirolimus, either with or without surface treatment, that is, the coating of a hydrophilic primer layer as described above in Example 2. The drug content on these balloons is 1.7 μm/mm² before folding. The amount of drug loss caused by the folding process is compared.

As illustrated in FIG. 7A(i), in the absence of surface treatment of the balloon surface with a hydrophilic primer layer, the drug on the balloon is measured at 0.95 μm/mm² following folding of the balloon using a standard automatic folding machine such as MSI FFS1075S. The reduction of drug concentration from 1.7 μm/mm² to 0.95 μm/mm² represents a loss of approximately 43.96% as demonstrated.

On the other hand, as illustrated in FIG. 7A(ii), when the exemplary balloon is prepared according to embodiment 1B of the present disclosure (i.e. with surface treatment by a hydrophilic primer layer), the drug content on the balloon is measured at 1.59 μm/mm² after folding. The reduction of drug concentration from 1.7 μm/mm² to 1.59 μm/mm² represents a much smaller reduction in drug content of 6.52%.

The above comparison clearly demonstrates the importance of the hydrophilic primer layer in retaining the drug on the balloon and such an effect is shown to be able to withstand the folding action of the balloon. The importance of the surface treatment of the surface of the balloon in retaining the drug even after folding is further demonstrated in FIG. 7B, which depicts various concentrations of sirolimus and polymers before and after folding, showing that there is a relatively low loss of drug from the catheter balloons even after folding has taken place regardless of the amount of drug concentration used in prepared the drug eluted balloons.

### Example 5—Effect of Drug Concentration on Elution Ratio

As can be seen by reference to FIG. 8A, a number of different concentrations of sirolimus in combination together with PLGA49700 have been prepared in accordance with the arrangement of layers depicted in FIG. 1B. The elution rate of the drug with respect to the ratio of the drug sirolimus with respect to the polymer is determined by using a drug elution test performed in PBS solution with shaking instrument at a shaking speed of 120 RPM and 37° C.

Similarly, as depicted in FIG. 8B a number of different combinations of sirolimus in combination together with PLGA 14300 have also been prepared in accordance with the arrangement of layers depicted in FIG. 1B. Again, the elution rate of the drug with respect to the ratio of the drug sirolimus with respect to the polymer is determined in accordance with the same drug elution test as described above.

The drug coated balloon was immersed in the 50 mM PBS at 37° C. The drug coated balloon elution system was subject to shaking at a speed of 120 RPM. The sample solution was then collected for HPLC analysis at time 1 min, 3 min and 10 min.

### Example 6 Analysis of Structural Characteristics of Balloon

An exemplary balloon prepared according to the schematic arrangement depicted in FIG. 1B after folding is shown in FIG. 10A. In this example, the balloon has been soaked and subjected to shaking for 2 minutes in PBS buffer solution (×20) at 120 RPM shaking.

An exemplary balloon prepared according to the schematic arrangement depicted in FIG. 1B in its unfolded state is shown in FIG. 10A. In this example, the balloon has been soaked in PBS buffer and subjected to shaking for 2 mins at 37 centigrade and further inflated at 8 atm pressure at 20 times magnitude.

FIG. 10C(i) and FIG. 10C(ii) show the balloon as illustrated in FIG. 10B in ×50 magnification and ×200 magnification respectively.

### Example 7—Expanding Coated Balloon in Artificial Heart Vessels

The in vitro experimental arrangement depicted in FIG. 11A and FIG. 11B is used to simulate the in vivo release of the pharmaceutically active agent into a vascular space by incubating the balloon which is coated with a pharmaceutically active agent (Sirolimus) in accordance with the embodiment depicted in FIG. 1B with three layers, whereby dextrin is used as the inclusion compound and PVP-based hydrophilic layer as described above is included.

A simulated blood vessel prepared from silica is fixed between plates before being filled with PBS or similar with a pH +7.4 (+/−0.2).

A guide wire is threaded through the simulated blood vessel and an exemplary catheter balloon in an aqueous bath including a buffer such as phosphate buffered saline (PBS) is introduced.

The release of the pharmaceutically active agent to the aqueous bath (e.g., the buffer) after expansion of the balloon is then assayed. Release profiles, both absolute w/w as well as kinetic J=−DdC/dx (see discussion below), of the pharmaceutical active agent are measured.

The concentration of the pharmaceutically active agent in the aqueous environment may be measured using any means, including, but not limited to, high pressure liquid chromatography (HPLC) or specific immunological assays.

For example, the amount of the pharmaceutically active agent released through the expandable cover within about 1 hour when incubated at about 20-25° or about 37° C. in an aqueous environment such as PBS, plasma, blood, a body fluid, or other aqueous medium is assayed.

Various time points may be used to assess release of the pharmaceutically active agent when the balloon is in an unexpanded state or an expanded state, including, but not limited to, within about 30 seconds, within about 1 minute, within about 2 minutes, within about 3 minutes, within about 4 minutes, within about 5 minutes, within about 6 minutes, within about 8 minutes, within about 9 minutes, within about 10 minutes, within about 15 minutes, within about 20 minutes, within about 25 minutes, within about 30 minutes, within about 35 minutes, within about 40 minutes, within about 45 minutes, within about 50 minutes, within about 55 minutes, within about 1 hour, within about 2 hours, within about 3 hours, within about 4 hours, within about 5 hours, within about 6 hours, within about 7 hours, within about 8 hours, within about 1-10 minutes, within about 10-100 minutes or within about 50-200 minutes.

As shown in FIG. 11C(i) and (ii), the drug/polymer is transferred from the drug eluted balloon to the simulated blood vessel. The drug content amounts are depicted graphically in FIG. 8C.

FIG. 12A demonstrates the content of sirolimus in simulated blood vessel after incubation of balloons formed of different concentrations of dextrin and sirolimus. In FIG. 12A, “150×1-6-4-3” refers to 150 mg/ml of dextrin, the number “6” refers to concentration of 6 mg/ml of dextrin in the first layer of drug coating solution, the number “4” refers to the concentration of 4 mg/ml of dextrin in the second layer of drug coating solution, and the number “3” refers to the concentration of 3 mg/ml of dextrin in the third layer of drug coating solution. The concentration of the drug is 3 mg/ml and the concentration of the polymer is 1.5 mg/ml.

When the experiments were repeated with pacitaxel, similar results as shown in the below table have been obtained, confirming the effective delivery of drug to the simulated blood vessel by the balloon of the present invention.

| | | Average amount |
|---|---|---|
| | Sample | of Paclitaxel |
| | No. | (ug) |
| Amount of Drug on Simulated Blood Vessel | 1 | 74.27 |
| | 2 | |
| Amount of Drug on Balloon (after delivery) | 1 | 109.58 |
| | 2 | |
| Amount of Drug originally provided on | 1 | 250.84 |
| Balloon | 2 | |

Further experiments were also conducted using simulated heart blood vessel based on a balloon prepared by dextrin of different concentrations to simulate the amount of medicine in blood vessels. These results are depicted graphically in FIG. 12B.

The above embodiments are described by way of example only. Many variations are possible without departing from the scope of the disclosure as defined in the appended claims.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

1. A balloon catheter comprising:
an expandable portion on an elongated body having a coating extending substantially about the external outer surface of at least the expandable portion;
wherein the coating comprises:
a primer layer on the surface of the expandable portion comprising at least one or more substantially hydrophilic compounds,
a first layer substantially overlying the primer layer and comprising an inclusion compound;
a second layer substantially overlying the first layer and comprising a mixture of a hydrophobic polymer, an active therapeutic agent and the inclusion compound.

2. The balloon catheter according to claim 1 wherein the active therapeutic agent of the top layer is selected from the group comprising sirolimus, everolimus, paclitaxel and taxol and including derivatives, hydrates, esters, salts, polymorphs or analogs thereof.

3. The balloon catheter according to claim 1 wherein the mass ratio of inclusion compound and hydrophobic polymer is 1:1-1:10,

4. The balloon catheter according to claim 1 wherein and the total loading of the active therapeutic agent of the coating of the surface of the balloon is 1-3 μg/mm²

5. The balloon catheter according to claim 1 wherein the at least one or more hydrophilic compound(s) of the primer layer is/are selected from the group comprising polyurethane acrylate, polyvinylpyrrolidone, polyvinyl alcohol, polyethylene glycol and polyacrylic acid.

6. The balloon catheter according to claim 1 wherein the second layer comprises a homogeneous mixture of the hydrophobic polymer, an active therapeutic agent and the inclusion compound.

7. The balloon catheter according to claim 1 wherein the second layer comprises microspheres having the active therapeutic agent encapsulated therein.

8. The balloon catheter according to claim 1 wherein the inclusion compound is chitosan, β-cyclodextrin, hydroxypropyl-β-cyclodextrin, hydroxypropyl-γ-cyclodextrin, hydroxyethyl cellulose, carboxymethyl cellulose, iopromide and derivatives thereof and dextran.

9. The balloon catheter according to claim 1 wherein the said the hydrophobic polymer carrier is selected from polylactic acid, polylactide hexalactide and polyglycolide lactide and has a molecular weight of 1-100 kDa.

10. A method of coating a balloon catheter comprising:
applying to the surface of a balloon catheter a first solution comprising at least one more of the group consisting of from ethanol, acetone, isopropanol, propanol, ethyl acetate, dichloromethane, tetrahydrofuran, acetonitrile and one or more hydrophilic compounds selected from the group consisting of polyurethane acrylate, polyvinylpyrrolidone, polyvinyl alcohol, polyethylene glycol and polyacrylic acid,
curing the coated catheter under hot air or ultraviolet light,
applying to the coated catheter a second solution comprising at least one or more solvents in the group comprising ethanol, water, acetone and isopropanol into which an inclusion compound has been dissolved;
drying the coated catheter and
applying to the coated catheter a third solution comprising a therapeutic compound, and a polymer solution in a solvent, said solvent selected from the group comprising ethanol, water, acetone, isopropanol, dichloromethane, trichloromethane and tetrahydrofuran.

11. The method of coating a balloon catheter according to claim 10 wherein the applying of the solutions to the catheter is performed by dipping, spraying or brushing the solutions thereon.

12. The method of coating a balloon catheter according to claim 10 wherein microspheres are formed by aggregation of the therapeutic compound, the polymer and the inclusion compound.
